(19) United States
(12) Patent Application Publication
(10) Pub. No.: US 2025/0375171 A1
(43) Pub. Date: Dec. 11, 2025

(54) PET APPARATUS

(71) Applicant: HAMAMATSU PHOTONICS K.K., Hamamatsu-shi (JP)

(72) Inventors: Kento HAKAMATA, Shizuoka (JP); Hidemoto YAMAUCHI, Shizuoka (JP); Nakahiro SATOH, Shizuoka (JP)

(73) Assignee: HAMAMATSU PHOTONICS K.K., Hamamatsu-shi (JP)

(21) Appl. No.: 19/220,484

(22) Filed: May 28, 2025

(30) Foreign Application Priority Data
Jun. 7, 2024 (JP) 2024-092854

(51) Int. Cl. A61B 6/03; A61B 6/00; A61B 6/04; A61B 6/42

(52) U.S. Cl. CPC A61B 6/037; A61B 6/0492; A61B 6/425; A61B 6/547

(57) ABSTRACT

A PET apparatus includes a detector ring, a processing unit, a position adjustment unit, and a control unit. The position adjustment unit adjusts a position of the detector ring by moving the detector ring relative to a subject in a body axis direction. The processing unit repeatedly obtains and outputs a position or a position change amount of the subject relative to the detector ring based on a coincidence detection event in which any two radiation detectors out of a plurality of radiation detectors in the detector ring detect a pair of gamma-rays. The control unit instructs the position adjustment unit to adjust the position of the detector ring such that the subject is positioned within a predetermined range in the body axis direction in the measurement space based on the position or the position change amount of the subject output from the processing unit.

### CROSS-REFERENCE TO RELATED APPLICATION

This application is based upon and claims the benefit of priority from Japanese Patent Application No. 2024-092854, filed on Jun. 7, 2024, the entire contents of which are incorporated herein by reference.

### TECHNICAL FIELD

The present disclosure relates to a PET apparatus.

### BACKGROUND

A positron emission tomography apparatus (a PET apparatus) includes a detector ring in which a plurality of radiation detectors are arranged around a measurement space in which a subject into which a drug which is labeled with a positron emitting radioisotope (an RI source) is injected is placed, and reconstructs a tomographic image of the subject based on a coincidence detection event in which any two radiation detectors out of the plurality of radiation detectors detect a pair of gamma-rays generated by a positron which is emitted from the RI source in the subject.

The PET apparatus may be an apparatus for a whole body, and in addition, in some cases, may be an apparatus dedicated to a body part, such as a head, a breast, or the like. As compared with the whole body PET apparatus, the body part specific PET apparatus often achieves cost reduction by reducing a size of the detector ring in the apparatus.

As compared with the whole body PET apparatus, in the body part specific PET apparatus in which the size of the detector ring is reduced, the degree of difficulty of adjustment of a position of the subject by a medical radiologic technologist or the like who is an operator is large, and the above position adjustment requires skill. In addition, the PET apparatus in general has problems that a measurement time is as long as 20 minutes or more, that invasiveness is very high in the case of performing body restraint on the subject so that the subject does not move during the measurement for a long time, and that re-examination becomes a large burden on the subject due to an expensive drug, a radiation exposure, and the like.

As a technique of allowing motion of the subject without performing the body restraint on the subject to perform the measurement, there is known a technique of detecting the motion of the subject by monitoring a marker which is provided on the subject with a camera, and moving the detector ring following the motion of the subject based on the detection result, or correcting the measurement data based on the motion of the subject. However, it is not easy to install the camera in the small detector ring provided in the body part specific PET apparatus.

In a technique described in Non Patent Document 1, the motion of the subject is detected based on the coincidence detection event, and the measurement data is corrected based on the detected motion of the subject. The above technique does not require an additional device such as a camera, and thus, it is possible to apply the technique to the body part specific PET apparatus in this respect. By applying the above technique to the body part specific PET apparatus, it is possible to perform the measurement while allowing the motion of the subject without performing the body restraint on the subject.

Non Patent Document 1: Y. Iwao et al., “Data-Driven Adaptive Frame Division Motion Correction for Brain PET”, 2023 IEEE NSS MIC RTSD, [M-17-219], 2023

### SUMMARY

Even in the case in which the technique described in Non Patent Document 1 is applied to the body part specific PET apparatus, the subject may go out of an axial field of view of the detector ring, and it is not appropriate to use the measurement data acquired in the above period for reconstruction of the tomographic image. In the case in which the subject goes out of the axial field of view of the detector ring, data loss or measurement failure may occur, and the re-examination may be required. When the re-examination is required as described above, a large burden is imposed on the subject in terms of the cost, the radiation exposure, and the like.

An object of the present invention is to provide a PET apparatus which does not require an additional device such as a camera in a detector ring, can perform measurement while allowing motion without performing body restraint on a subject, and can avoid data loss and measurement failure.

An embodiment of the present invention is a PET apparatus. The PET apparatus includes (1) a detector ring provided with a plurality of radiation detectors arranged around a measurement space in which a subject into which a drug labeled with a positron emitting radioisotope is injected is placed; (2) a position adjustment unit for adjusting a position of the detector ring by moving the detector ring relative to the subject in a body axis direction; (3) a processing unit for repeatedly obtaining and outputting a position or a position change amount of the subject relative to the detector ring in the body axis direction based on a coincidence detection event in which any two radiation detectors out of the plurality of radiation detectors detect a pair of gamma-rays generated by a positron emitted from the positron emitting radioisotope in the subject; and (4) a control unit for instructing the position adjustment unit to adjust the position of the detector ring such that the subject is positioned within a predetermined range in the body axis direction in the measurement space based on the position or the position change amount of the subject output from the processing unit.

According to the PET apparatus of the embodiment of the present invention, an additional device such as a camera is not required in a detector ring, and it is possible to perform measurement while allowing motion without performing body restraint on a subject, and further, it is possible to avoid data loss and measurement failure. In addition, the PET apparatus according to the embodiment of the present invention can be applied to the whole body PET apparatus, and in particular, can be suitably applied to the body part specific PET apparatus.

The present invention will be more fully understood from the detailed description given hereinbelow and the accompanying drawings, which are given by way of illustration only and are not to be considered as limiting the present invention.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will be apparent to those skilled in the art from this detailed description.

### BRIEF DESCRIPTION OF THE DRAWINGS

FIG. 1 is a diagram illustrating a configuration of a PET apparatus 1.

FIG. 2A and FIG. 2B are diagrams illustrating a subject setting fixture 12 provided in a detector ring 10.

FIG. 3A to FIG. 3C are diagrams for describing an operation of a control unit 40 and a position adjustment unit 20.

FIG. 4 is a flowchart illustrating a first operation example of the control unit 40 and the position adjustment unit 20.

FIG. 5 is a flowchart illustrating a second operation example of the control unit 40 and the position adjustment unit 20.

### DETAILED DESCRIPTION

Hereinafter, embodiments of a PET apparatus will be described in detail with reference to the accompanying drawings. In the description of the drawings, the same elements will be denoted by the same reference signs, and redundant description will be omitted. The present invention is not limited to these examples, and the Claims, their equivalents, and all the changes within the scope are intended as would fall within the scope of the present invention.

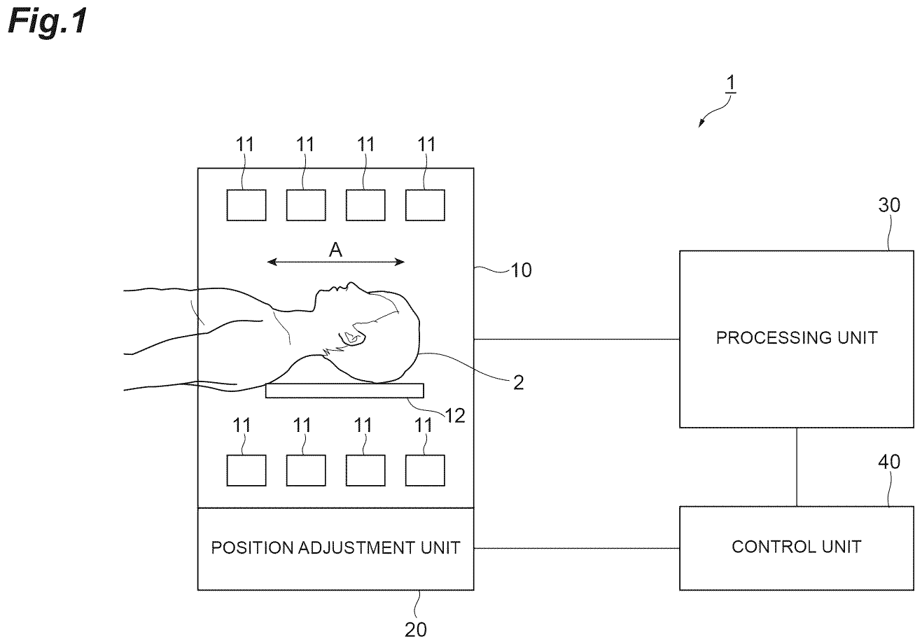

FIG. 1 is a diagram illustrating a configuration of a PET apparatus 1. The PET apparatus 1 includes a detector ring 10, a processing unit 30, a position adjustment unit 20, and a control unit 40. In this diagram, as the PET apparatus 1, a head specific PET apparatus in which a head of a human is set as a subject 2 is illustrated.

The detector ring 10 is provided with a plurality of radiation detectors 11 which are arranged around a measurement space in which the subject 2 into which a drug labeled with a positron emitting radioisotope (an RI source) is injected is placed. When a positron is emitted from the RI source in the subject 2, the positron immediately undergoes electron positron annihilation with a nearby electron to generate a pair of gamma-rays traveling in opposite directions.

In the case in which the pair of gamma-rays are detected in coincidence by any two radiation detectors 11 out of the plurality of radiation detectors 11 of the detector ring 10, the RI source from which the positron is emitted is present on a coincidence detection line (a line of response) connecting the two radiation detectors 11 to each other. By performing image reconstruction processing based on the information of the coincidence detection lines which are acquired for a large number of coincidence detection events, that is, the position information of each of the two radiation detectors 11 with which the pair of gamma-rays are detected in coincidence, it is possible to create a tomographic image representing an RI source distribution in the subject 2.

Further, in the configuration illustrated in FIG. 1, the detector ring 10 includes a subject setting fixture 12 which is provided with a marker for positioning the subject 2 within a predetermined range in a body axis direction A at the time of a measurement start. In the case in which the subject 2 is set to the head, the subject setting fixture 12 may be a headrest on which the head is placed.

The position adjustment unit 20 adjusts a position of the detector ring 10 by moving the detector ring 10 relative to the subject 2 in the body axis direction A. The position adjustment unit 20 may be configured to include, for example, a programmable logic controller (PLC) and a servo motor. The PLC receives a control signal output from the control unit 40, and moves the detector ring 10 along the body axis direction A by driving the servo motor according to a direction and a movement amount which are indicated by the control signal.

The processing unit 30 creates the tomographic image of the subject 2 by performing the image reconstruction processing as described above, and in addition, performs the following processing.

The processing unit 30 repeatedly obtains a position or a position change amount of the subject 2 relative to the detector ring 10 in the body axis direction A based on the coincidence detection event in which any two radiation detectors 11 out of the plurality of radiation detectors 11 detect the pair of gamma-rays generated by the positron which is emitted from the RI source in the subject 2, and repeatedly outputs the position or the position change amount to the control unit 40. In addition, the position change amount is set to a positive value or a negative value according to a direction of a motion of the subject 2. Further, in the case in which there is no motion of the subject 2, the position change amount is set to 0.

For example, the processing unit 30 may obtain an average position of respective center points of the plurality of coincidence detection lines which are acquired in a certain predetermined period, and may use the above average position as the information of the position of the subject 2 in the predetermined period, and further, may use a difference between the above average position and the average position of the respective center points of the plurality of coincidence detection lines which are acquired in the previous predetermined period as the position change amount of the subject 2.

The processing unit 30 may obtain and output the position or the position change amount of the subject 2 for each of the predetermined periods.

Further, the processing unit 30 may calculate the position change amount from a difference between a previous image and a subsequent image. For example, the processing unit 30 may create the tomographic image of the subject 2 by performing the image reconstruction processing based on the plurality of coincidence detection lines which are acquired in the certain predetermined period, and may use a center of gravity position in the body axis direction A in the tomographic image as the information of the position of the subject 2 in the predetermined period, and further, may use a difference between the above center of gravity position and the center of gravity position in the body axis direction A in the tomographic image which is acquired in the previous predetermined period as the position change amount of the subject 2.

In this case also, the processing unit 30 may obtain and output the position or the position change amount of the subject 2 for each of the predetermined periods.

Further, for example, the processing unit 30 may obtain the position or the position change amount of the subject 2 for each of the predetermined periods as described above, accumulate the position change amounts, and may output the position or the position change amount of the subject 2 when the position change amount accumulated as described above exceeds a certain range. In this case, in the case in which the motion of the subject 2 is large in the body axis direction A, the frequency of the output of the position or the position change amount of the subject 2 is high, and in the case in which the motion of the subject 2 is small in the body axis direction A, the frequency of the output of the position or the position change amount of the subject 2 is low.

The control unit 40 instructs the position adjustment unit 20 to adjust the position of the detector ring 10 such that the subject 2 is positioned within the predetermined range in the body axis direction A in the measurement space based on the position or the position change amount of the subject 2 which is output from the processing unit 30.

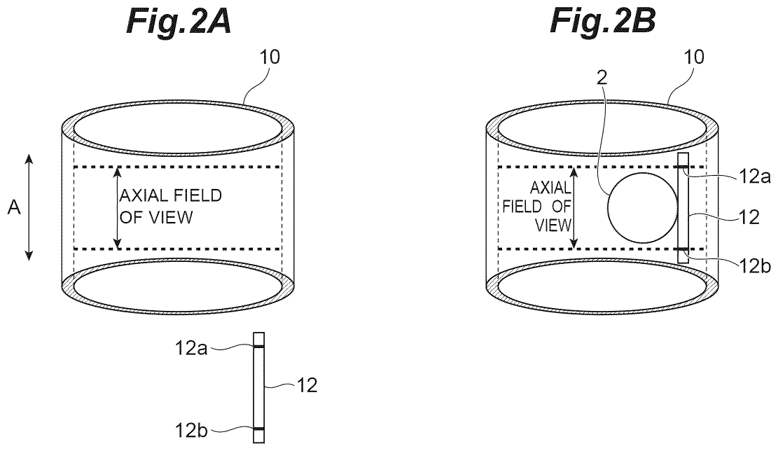

FIG. 2A and FIG. 2B are diagrams illustrating the subject setting fixture 12 which is provided in the detector ring 10. As illustrated in FIG. 2A, the markers 12a and 12b for positioning the subject 2 within the predetermined range in the body axis direction A at the time of the measurement start are provided on the subject setting fixture 12. In this case, the predetermined range in the body axis direction A may be a range of the body axial field of view, or may be a range of a part of the body axial field of view.

A medical radiologic technologist or the like as an operator places the subject 2 (in particular, a portion including a region for which the tomographic image is desired to be acquired) between the positions to which the markers 12a and 12b are respectively provided on the subject setting fixture 12, and places the subject setting fixture 12 and the subject 2 in the measurement space of the detector ring 10 in the above state as illustrated in FIG. 2B. By using the above configuration, it is possible to reliably place the subject 2 in the body axial field of view at the time of the measurement start.

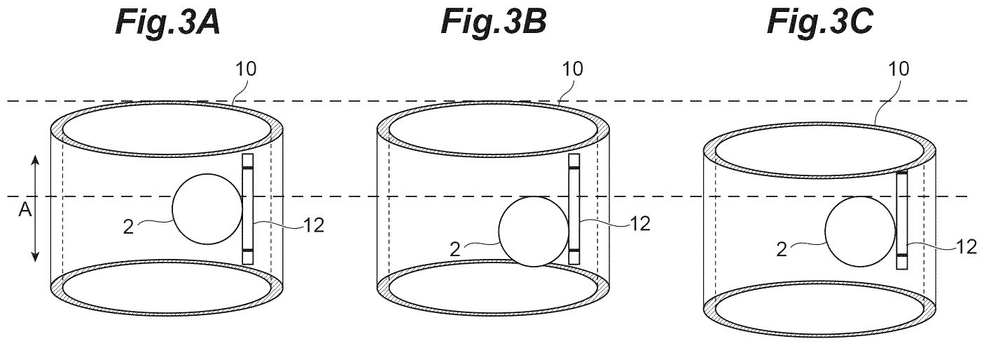

FIG. 3A to FIG. 3C are diagrams for describing the operation of the control unit 40 and the position adjustment unit 20. Even in the case in which the subject 2 is placed in the axial field of view at the time of the measurement start (FIG. 3A), when the subject 2 moves in the body axis direction A during the measurement, all or a part of the subject 2 may deviate from the axial field of view (FIG. 3B). In the case in which the subject 2 goes out of the axial field of view, the data loss or the measurement failure may occur, and the re-examination may be required in some cases.

In consideration of the above, in the present embodiment, the control unit 40 instructs the position adjustment unit 20 to perform the adjustment of the position of the detector ring 10 such that the subject 2 is positioned within the predetermined range in the body axis direction A in the measurement space based on the position or the position change amount of the subject 2 which is output from the processing unit 30. By using the above configuration, even in the case in which the subject 2 moves in the body axis direction A during the measurement, it is possible to prevent the subject 2 from deviating from the body axial field of view (FIG. 3C).

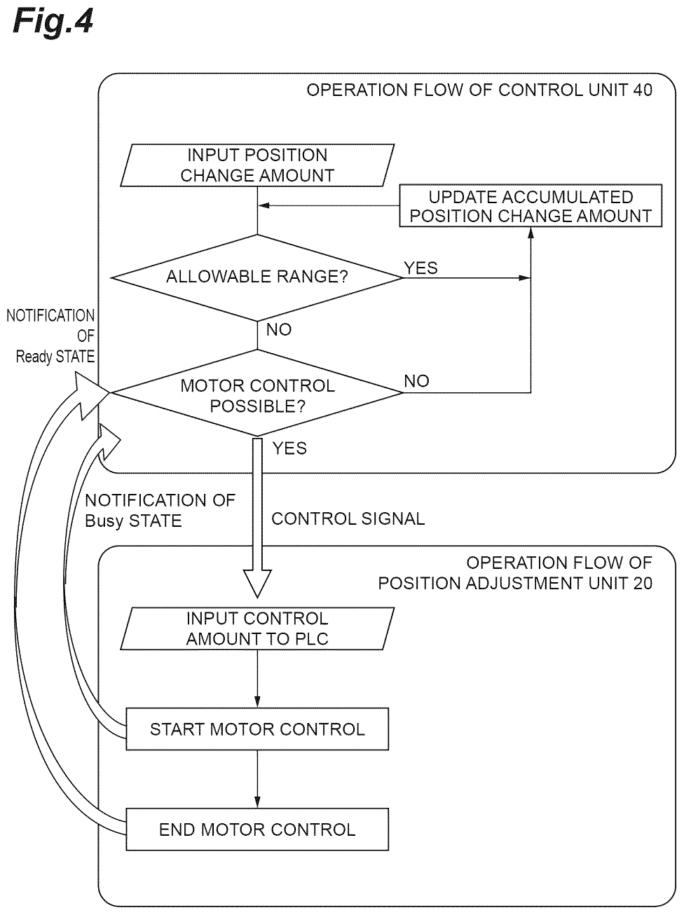

FIG. 4 is a flowchart illustrating a first operation example of the control unit 40 and the position adjustment unit 20. In the first operation example in FIG. 4, the control unit 40 initializes the accumulated position change amount Dsum of the subject 2 at the time of the measurement start, and then performs the following processing each time the position or the position change amount of the subject 2 is output from the processing unit 30.

When the position change amount D of the subject 2 output from the processing unit 30 is input, the control unit 40 adds the position change amount D to the accumulated position change amount Dsum to update the accumulated position change amount Dsum of the subject 2. Further, when the position of the subject 2 output from the processing unit 30 is input, the control unit 40 adds the position change amount D which is obtained based on the above position to the accumulated position change amount Dsum to update the accumulated position change amount Dsum of the subject 2.

In addition, in the case in which the accumulated position change amount Dsum is not within an allowable range and the movement start of the detector ring 10 by the position adjustment unit 20 is possible (in the case in which the control of the servo motor of the position adjustment unit 20 is possible), the control unit 40 outputs, to the position adjustment unit 20, the control signal to instruct the position adjustment unit 20 to move the detector ring 10 according to the accumulated position change amount Dsum of the subject 2, and further, initializes the accumulated position change amount Dsum.

The position adjustment unit 20 receives the control signal from the control unit 40, and drives the servo motor according to the direction and the movement amount which are indicated by the control signal. At the time of the control start of the servo motor, the position adjustment unit 20 notifies the control unit 40 that the servo motor is in a Busy state in which it is not possible to control the servo motor. Further, at the time of the control end of the servo motor, the position adjustment unit 20 notifies the control unit 40 that the servo motor is in a Ready state in which it is possible to control the servo motor.

By receiving the notification of the Busy state or the Ready state from the position adjustment unit 20, the control unit 40 can recognize whether or not the movement start of the detector ring 10 by the position adjustment unit 20 is possible (whether or not it is possible to control the servo motor of the position adjustment unit 20).

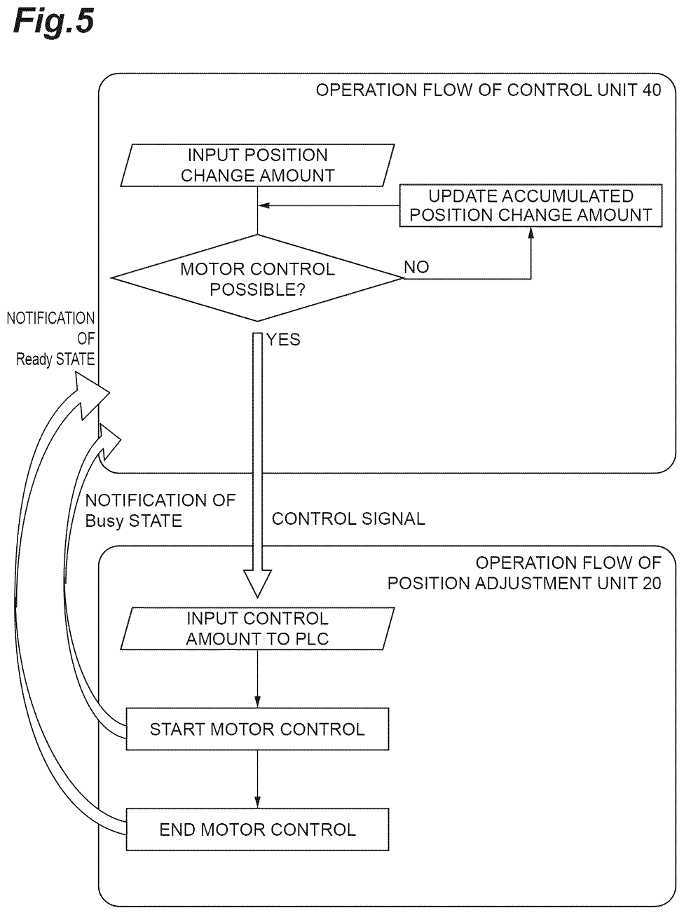

FIG. 5 is a flowchart illustrating a second operation example of the control unit 40 and the position adjustment unit 20. As compared with the first operation example in FIG. 4, the second operation example in FIG. 5 is different in that the control unit 40 does not determine whether or not the accumulated position change amount Dsum is within the allowable range.

That is, in the case in which the movement start of the detector ring 10 by the position adjustment unit 20 is possible (in the case in which the control of the servo motor of the position adjustment unit 20 is possible), the control unit 40 outputs, to the position adjustment unit 20, the control signal to instruct the position adjustment unit 20 to move the detector ring 10 according to the accumulated position change amount Dsum of the subject 2, and further, initializes the accumulated position change amount Dsum.

In the first operation example of FIG. 4, the frequency of the movement of the detector ring 10 is low, and the movement amount when moving the detector ring 10 is large. On the other hand, in the second operation example of FIG. 5, the frequency of the movement of the detector ring 10 is high, and the movement amount when moving the detector ring 10 is small.

In addition, the technique described in Non Patent Document 1 may be additionally used in combination. In particular, in the first operation example of FIG. 4, the accumulation of the motion of the subject 2 increases in the period until the detector ring 10 is moved, and thus, it is preferable to apply the technique described in Non Patent Document 1 and correct the measurement data based on the motion of the subject 2 during the above period.

As described above, according to the PET apparatus of the present embodiment, an additional device such as a camera is not required in the detector ring 10, and it is possible to perform the measurement while allowing the motion of the subject 2 without performing the body restraint on the subject 2, and further, it is possible to avoid the data loss and the measurement failure. The PET apparatus according to the present embodiment can be used as the whole body PET apparatus, and in addition, in particular, the PET apparatus can be suitably used as the body part specific PET apparatus in which the detector ring 10 is small.

The PET apparatus is not limited to the embodiments and configuration examples described above, and various modifications are possible.

The PET apparatus of a first aspect according to the above embodiment is a PET apparatus including (1) a detector ring provided with a plurality of radiation detectors arranged around a measurement space in which a subject into which a drug labeled with a positron emitting radioisotope is injected is placed; (2) a position adjustment unit for adjusting a position of the detector ring by moving the detector ring relative to the subject in a body axis direction; (3) a processing unit for repeatedly obtaining and outputting a position or a position change amount of the subject relative to the detector ring in the body axis direction based on a coincidence detection event in which any two radiation detectors out of the plurality of radiation detectors detect a pair of gamma-rays generated by a positron which is emitted from the positron emitting radioisotope in the subject; and (4) a control unit for instructing the position adjustment unit to adjust the position of the detector ring such that the subject is positioned within a predetermined range in the body axis direction in the measurement space based on the position or the position change amount of the subject which is output from the processing unit.

In the PET apparatus of a second aspect, in the configuration of the first aspect, the control unit may update an accumulated position change amount of the subject each time the position or the position change amount of the subject is output from the processing unit, and in a case in which the accumulated position change amount is not within an allowable range and a movement start of the detector ring by the position adjustment unit is possible, the control unit may instruct the position adjustment unit to move the detector ring according to the accumulated position change amount of the subject, and may initialize the accumulated position change amount.

In the PET apparatus of a third aspect, in the configuration of the first aspect, the control unit may update an accumulated position change amount of the subject each time the position or the position change amount of the subject is output from the processing unit, and in a case in which a movement start of the detector ring by the position adjustment unit is possible, the control unit may instruct the position adjustment unit to move the detector ring according to the accumulated position change amount of the subject, and may initialize the accumulated position change amount.

In the PET apparatus of a fourth aspect, in the configuration of any one of the first to third aspects, the detector ring may include a subject setting fixture including a marker for positioning the subject within the predetermined range in the body axis direction at a time of a measurement start.

The present invention can be used as a PET apparatus which does not require an additional device such as a camera in a detector ring, can perform measurement while allowing motion without performing body restraint on a subject, and can avoid data loss and measurement failure.

From the invention thus described, it will be obvious that the invention may be varied in many ways. Such variations are not to be regarded as a departure from the spirit and scope of the invention, and all such modifications as would be obvious to one skilled in the art are intended for inclusion within the scope of the following claims.

What is claimed is:

1. A PET apparatus comprising:
a detector ring provided with a plurality of radiation detectors arranged around a measurement space in which a subject into which a drug labeled with a positron emitting radioisotope is injected is placed;
a position adjustment unit configured to adjust a position of the detector ring by moving the detector ring relative to the subject in a body axis direction;
a processing unit configured to repeatedly obtain and output a position or a position change amount of the subject relative to the detector ring in the body axis direction based on a coincidence detection event in which any two radiation detectors out of the plurality of radiation detectors detect a pair of gamma-rays generated by a positron emitted from the positron emitting radioisotope in the subject; and
a control unit configured to instruct the position adjustment unit to adjust the position of the detector ring such that the subject is positioned within a predetermined range in the body axis direction in the measurement space based on the position or the position change amount of the subject output from the processing unit.

2. The PET apparatus according to claim 1, wherein the control unit is configured to update an accumulated position change amount of the subject each time the position or the position change amount of the subject is output from the processing unit, and
in a case in which the accumulated position change amount is not within an allowable range and a movement start of the detector ring by the position adjustment unit is possible, the control unit is configured to instruct the position adjustment unit to move the detector ring according to the accumulated position change amount of the subject, and initialize the accumulated position change amount.

3. The PET apparatus according to claim 1, wherein the control unit is configured to update an accumulated position change amount of the subject each time the position or the position change amount of the subject is output from the processing unit, and
in a case in which a movement start of the detector ring by the position adjustment unit is possible, the control unit is configured to instruct the position adjustment unit to move the detector ring according to the accumulated position change amount of the subject, and initialize the accumulated position change amount.

4. The PET apparatus according to claim 1, wherein the detector ring includes a subject setting fixture including a marker in order to position the subject within the predetermined range in the body axis direction at a time of a measurement start.
